(19) United States
(12) Patent Application Publication
(10) Pub. No.: US 2022/0146398 A1
(43) Pub. Date: May 12, 2022

(54) Multi-ion sensor, flow measuring cell and system for measuring ions in aqueous systems

(71) Applicant: Matthias Groß, Kaiserslautern (DE)

(72) Inventor: Matthias Groß, Kaiserslautern (DE)

(21) Appl. No.: 17/521,927

(22) Filed: Nov. 9, 2021

(30) Foreign Application Priority Data
Nov. 10, 2020 (DE) 10 2020 129 629.0

(51) Int. Cl. G01N 15/06; G01N 33/18

(52) U.S. Cl. CPC G01N 15/0656; G01N 33/18

(57) ABSTRACT

The present invention relates to a multi-ion sensor (1) for measuring ions in aqueous systems comprising
a flexible or semi-flexible substrate (11),
a reference electrode (13) applied to the substrate (11),
at least three working electrodes (15a, 15b, 15c) applied to the substrate (11),
   wherein the substrate (11), the reference electrode (13) and the working electrodes (15a, 15b, 15c) form an ion-selective sensor unit (101),
a connection device (17) for the ion-selective sensor unit (101) and
a sensor housing (19) which accommodates an assembly of ion-selective sensor unit (101) and connection device (17),
wherein the reference electrode (13) has dimensions of max. 4 mm×4 mm×4 mm.

The present invention is furthermore directed to a flow measuring cell (3) for measuring ions in aqueous systems as well as to a respective method for measuring ions in aqueous systems and a system for measuring ions in aqueous systems and for adjusting the ion contents in the aqueous systems.

The present invention relates to a multi-ion sensor, a flow measuring cell and a system for measuring ions in aqueous systems, and to a corresponding method.

Generic multi-ion sensors and corresponding systems for measuring ions in aqueous systems are generally known from the prior art. All these known devices have in common that they have individual electrodes in so-called “solid state” technology, in particular in the form of carbon tubes, for each ion to be determined and for the reference, so that the actual multi-ion sensor is very large and expensive due to the plurality of individual electrodes. Furthermore, this type of multi-ion sensor requires intermediate cleaning of the electrodes during the measurement, e.g. with distilled water. Finally, both sample and reference liquids are usually pumped into an open container in which the generic multi-ion sensor is inserted, so the volume of liquid to be measured is relatively large. Finally, the liquid has to be pumped out again after the calibrations and after the measurement and from the container and generates a not small waste volume.

There is therefore a need for a novel multi-ion sensor and a corresponding measurement system that can overcome the disadvantages of the prior art.

The present invention is therefore based on the task of providing a multi-ion sensor, a flow measuring cell and a system for measuring ions in aqueous systems, and of specifying a corresponding method, which are more cost-effective than the known systems and at the same time require less measuring volume. A further aim is a more precise measurement.

This task is solved in a first aspect of the present invention by a multi-ion sensor (1) for measuring ions in aqueous systems, comprising
a flexible or semi-flexible substrate (11),
a reference electrode (13) applied to the substrate (11)
at least three working electrodes (15a, 15b, 15c) applied to the substrate (11),
   wherein the substrate (11), the reference electrode (13) and the working electrodes (15a, 15b, 15c) form an ion-selective sensor unit (101),
a connection device (17) for the ion-selective sensor unit (101) and
a sensor housing (19) which accommodates an assembly of ion-selective sensor unit (101) and connection device (17),
wherein the reference electrode (13) has dimensions of max. 4 mm×4 mm×4 mm.

By “multi-ion sensor” it is understood according to the invention that the sensor is designed to measure different ions (i.e. cations and anions) in an aqueous system.

According to the invention, the substrate (11) is designed to be flexible or semi-flexible, which is ensured by using, for example, a thin flexible printed circuit board.

The reference electrode (13) has a constant equilibrium potential which adjusts quickly and reproducibly and serves as a reference point for measuring the relative potentials of the working electrodes (15a, 15b, 15c).

The working electrodes (15a, 15b, 15c) used according to the invention are directed to specific ions (cations and anions), whereby electrochemical processes take place in a controlled manner on their surfaces, the potential of which is determined in comparison to the reference electrode (13).

The unit referred to in the invention as the ion-selective sensor unit (101) comprises the reference electrode (13) and the working electrodes (15a, 15b, 15c), which are applied to the substrate (11).

According to the invention, the connection device (17) serves to receive and electrically contact the sensor unit (101). For this purpose, the sensor unit (101) may have a contact surface at the opposite end of the electrode ends, which is inserted into a corresponding receptacle of the connection device (17). Expediently, the connection device (17) further comprises a data cable provided with a plug at the end.

The sensor housing (19) essentially serves to protect the sensor unit (101) from moisture and mechanical influences. Its external dimensions are also adapted to the flow measuring cell (3).

Essential to the invention is the reference electrode (13), which has a minimum size of max. 4 mm×4 mm×4 mm compared to the prior art. The lower limit of the size of the reference electrode (13) is the technically feasible size at present and can be stated as 0.5 mm×0.5 mm×0.5 mm. The particularly preferred size is between 1.8 mm×1.8 mm×1.8 mm and 2.2 mm×2.2 mm×2.2 mm.

Moreover, the reference electrode (13) according to the invention has a precision superior to that of the prior art with respect to pseudo-reference electrodes, while requiring less effort, reduced size and lower cost with respect to true prior art reference electrodes.

Finally, the reference electrode (13) according to the invention is a true reference electrode and not a pseudo-reference electrode as mostly used in the prior art. With the true reference electrode (13) of the present invention, a constant potential is generated that is independent of the sample solution. Pseudo reference electrodes, on the other hand, are usually metallic conductors (e.g. as metal wires) that are immersed directly in an electrolyte solution/sample solution. Although a constant potential is also established at such pseudo reference electrodes, this is unknown and depends on the composition of the electrolyte solution/sample solution.

Within the scope of the multi-ion sensor (1), the present invention has the advantage of a significantly smaller construction compared to the known sensors, which is made possible by the printing of the working electrodes and the application of the real miniature reference electrode (13). In addition, the multi-ion sensor (1) according to the invention is significantly cheaper than a sensor consisting of the known solid-state single electrodes and a separate reference electrode.

It is further essential to the invention that the real reference electrode (13) (not a pseudo-reference electrode!) and the working electrodes (15a, 15b, 15c) in the miniaturised form are arranged integrally on one component, in the present invention this integrated component is referred to as an ion-selective sensor unit (101).

In a further embodiment of the present multi-ion sensor (1), the material of the reference electrode (13) is selected from polymer-coated fibres and Ag/AgCl.

In one embodiment of the present multi-ion sensor (1) according to the invention, the sensor housing (19) accommodates the assembly of the ion-selective sensor unit (101) and the connection device (17) in such a way that the rear side of the substrate (11) rests on a supporting part (191) of the sensor housing (19), and in that the sensor housing (19) encloses the assembly of ion-selective sensor unit (101) and connection device (17) to such an extent that the ends of the reference electrode (13) and the working electrodes (15a, 15b, 15c) are exposed with respect to the outside and thus form the measuring head (103) of the multi-ion sensor (1).

This embodiment first ensures that the mechanically sensitive substrate (11) is sufficiently supported to be available for measurement.

The embodiment shown above can be further embodied in such a way that the sensor housing (19) has a recessed partial surface (193) which corresponds geometrically with the supporting part (191) with an ion-selective sensor unit (101) resting thereon, so that a measuring gap (S) is formed between the ion-selective sensor unit (101) on the supporting part (191) and the recessed partial surface (193).

This embodiment with the creation of a measuring gap (S) improves the flow of the liquid to be measured over the ion-selective sensor unit (101).

A particular embodiment provides for a two-part sensor housing (19),
wherein a first sensor housing half accommodates the assembly of ion-selective sensor unit (101) and connection device (17) such that the rear side of the substrate (11) rests on a supporting part (191) of the first sensor housing half,
wherein a second sensor housing half encloses the assembly of ion-selective sensor unit (101) and connection device (17) to such an extent that the ends of the reference electrode (13) and of the working electrodes (15a, 15b, 15c) are exposed with respect to the outside and thus form the measuring head (103) of the multi-ion sensor (1).

It has been found to be particularly advantageous if the height (H) of the measuring gap (S) and the width (B) of the measuring gap (S) are in a geometric ratio H:B of 1:1 to 4:1, since in this way a laminar flow is generated in the measuring gap (S).

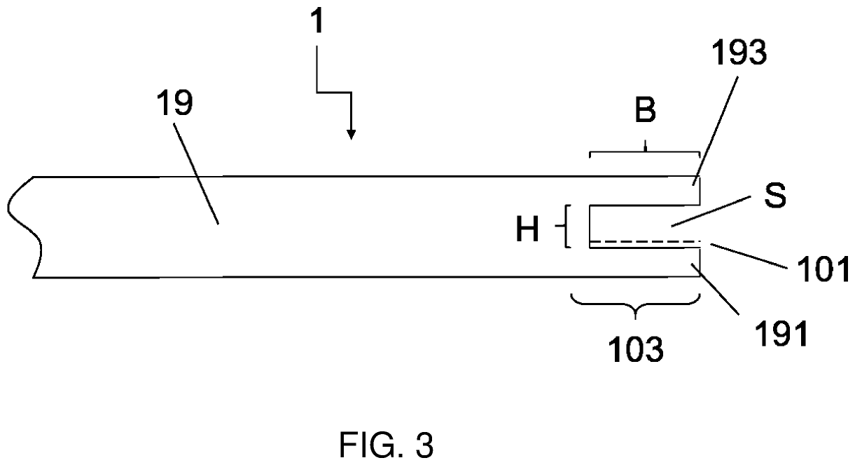

The height (H) of the measuring gap (S) results from the distance between the ion-selective sensor unit (101) resting on the supporting part (191) and the recessed partial surface (193). The width (B) of the measuring gap (S) corresponds to the open dimension in the longitudinal direction of the multi-ion sensor (1), which is shown in FIG. 3.

In a second aspect of the present invention, the aforementioned problem is solved by a flow measuring cell (3) for measuring ions in aqueous systems, which comprises
a receiving opening (31) for mounting a multi-ion sensor (1) according to the invention, as described above,
   wherein the measuring head (103) of the multi-ion sensor (1) can be mounted positively in the receiving opening (31) and together with the rear wall of the receiving opening (31) can form a flow measuring chamber,
at least one calibration supply line (33),
at least one sample supply line (35),
a collecting line (37) receiving the at least one calibration supply line (33) and the at least one sample supply line (35) and extending towards the receiving opening (31)
a drain line (39) which connects upstream of the receiving opening (31).

The receiving opening (31) is in particular a milled out part whose contour corresponds to the outer contour of the multi-ion sensor (1) so that it can be positively recessed. The ends of the measuring head (103) rest against the rear wall of the receiving opening (31), so that the flow measuring space is formed, which consists mainly of the measuring gap (S) and the supply lines leading directly into it.

For further securing, in particular against twisting, the measuring head (103) of the multi-ion sensor (1) can have lateral grooves in which suitable guide rails engage, which are provided on the inside of the receiving opening (31). Securing against rotation is particularly important to ensure that the measuring gap (S) is precisely adjusted to form a laminar flow.

The calibration supply line (33) and the sample supply line (35) are in particular bores/milled holes, at the outer end of which corresponding connections (e.g. hose sockets) are provided. At their inner end, the supply lines (33, 35) lead into the collecting line (37), which is aligned towards the receiving opening (31).

In particular, the collecting line (37) opens into the lower area of the receiving opening (31), so that the liquid passing through is introduced from below into the flow measuring space, or into the measuring gap (S), and can form a laminar flow.

The drain line (39) is also in particular a bore/milled hole, which is advantageously arranged above the flow measuring chamber or the measuring gap (S).

As a special feature, a widened cavity can be provided at the transition from the receiving opening (31) to the drain line (39) in order to collect any air bubbles that may occur and thus not falsify the precise measurement.

The flow measuring cell (3) according to the invention has the advantage that it is not an open container that has to be filled and emptied again. Influences of the ambient air as well as evaporation and associated concentration fluctuations are thus prevented. Furthermore, the flow measuring cell (3) according to the invention has a minimum volume and thus very small flow dead spaces, and self-venting can be provided. These features lead to the possibility of carrying out measurements with high measuring accuracy at a small sample volume and simple 2-point calibration. Finally, no intermediate flushing is necessary according to the invention.

A further development of the flow measuring cell (3) according to the invention provides that the diameters of the calibration supply line (33) and/or the sample supply line (35) and/or the collecting line (37) are 2 mm to 4 mm.

This achieves a significantly reduced volume for the flow measuring cell (3) compared to the state of the art, both for the sample and for the calibration liquids. On the one hand, this contributes to the economical use of the liquids used for the references, and on the other hand, the multi-ion sensor (1) according to the invention is designed to measure extremely precisely even with very small volumes.

In particular, the cumulative volume of the calibration supply line (33), the sample supply line (35), the collecting line (37) and the flow measurement chamber is 2 ml to 5 ml.

A third aspect of the present invention for solving the above problem relates to a method for measuring ions in aqueous systems, comprising the steps of
a) providing a flow measuring cell (3) according to the invention, as described above, with an assembled multi-ion sensor (1) according to the invention, as described above,
b) introducing a first aqueous calibration solution into the flow measuring cell (3) with flushing of the ion-selective sensor unit (101) of the multi-ion sensor (1), thereby carrying out a first calibration and recording a first reference measured value,
c) introducing an aqueous sample into the flow measuring cell (3) with flushing of the ion-selective sensor unit (101) of the multi-ion sensor (1), thereby recording a measurement value bundle,
d) introducing a second aqueous calibration solution into the flow measuring cell (3) with flushing of the ion-selective sensor unit (101) of the multi-ion sensor (1), thereby carrying out a second calibration and recording a second reference measured value,
   wherein steps a), b) c) and d) are carried out quasi-continuously without draining the flow measuring cell (3),
e) calculating the measurement values from first and second reference measurement values and measurement value bundles,
f) processing the measured values in a computing unit.

In steps b), c) and d), the respective liquids are not actively drained, but displacement results automatically from the introduction of the next liquid. Due to the small cross-sections of the pipes and the associated small volumes, such a procedure is possible without the successive liquids mixing to any appreciable extent. For this reason, too, intermediate flushing can be dispensed with.

The first aqueous calibration solution may in particular be water with ion concentrations slightly below the expected concentrations.

In a particularly preferred embodiment, the aqueous sample is the water of an aquarium.

The second aqueous calibration solution may in particular be water with ion concentrations slightly above the expected concentrations.

For carrying out the invention, it has been found to be particularly advantageous if the concentration of the various ions in the sample range between the respective concentrations of the first and second aqueous calibration solutions.

During the measurement in step c), a measured value specific to an ion (anion or cation) is recorded by each of the individual working electrodes (15a, 15b, 15c), and the totality of these specific measured values results in the measured value bundle.

Within the scope of the method, the present invention has in principle the advantages already described above that the multi-ion sensor (1) according to the invention and the flow measuring cell (3) according to the invention enable a significantly more cost-effective measurement, compared to the prior art. The measurement is more precise due to the small volumes and is easier and simpler to perform.

In a further development of the method according to the invention, it has been found to be advantageous if for the method according to the invention the volume of the first aqueous calibration solution, the aqueous sample and the second aqueous calibration solution required for the measurement is 3 ml to 10 ml.

With such a rather small volume, a precise measurement is nevertheless made possible due to the small dimensions of the multi-ion sensor (1) and flow measuring cell (3) according to the invention.

It has been found to be advantageous for an even more precise measurement if, prior to step b), the volume of liquid present in the supply lines to the flow measuring cell (3) is completely replaced by fresh volume of liquid of the aqueous system, whereby the replaced volume of liquid present is supplied to the aqueous system again.

On the one hand, this provides fresh liquid to be measured, and on the other hand, this liquid is not discarded and thus wasted, which is cost-saving on the one hand and environmentally friendly on the other hand.

Finally, in a fourth aspect for solving the above problem, the present invention relates to a system for measuring ions in aqueous systems and for adjusting the ion contents in the aqueous systems, comprising
a flow measuring cell (3) according to the invention, as described above,
a multi-ion sensor (1) according to the invention, as described above, mounted in the flow measuring cell (3)
a pump unit (5), which is fluid-dynamically connected on the input side to the aqueous system and to storage containers for the first and second aqueous calibration solutions for taking the sample, and which is fluid-dynamically connected on the output side to the at least one calibration supply line (33) and the at least one sample supply line (35),
a control unit (7) which is electronically connected on the input side to the multi-ion sensor (1),
   the control unit (7) having evaluation electronics for processing the first and second reference measured values and measured value bundles.

The pump unit (5) has a multi-channel design and, according to the invention, has separately controllable metering pumps for small volumes. These dosing pumps can be controlled independently of each other.

According to the invention, the control unit is, for example, an aquarium computer which processes information from the measurements of ion concentrations and regulates the ion concentration via suitable measures (e.g. changing the ion supply by controlling dosing pumps or solenoid valves) depending on the control difference (setpoint to actual) of the ion concentration.

The system according to the invention has in principle the advantages already described above, namely that with the multi-ion sensor (1) according to the invention and the flow measuring cell (3) according to the invention, a significantly more cost-effective measurement is made possible compared to the prior art. The measurement is more precise due to the small volumes and is easier and simpler to perform.

A further development of the system provides that the control/regulation unit is designed to regulate the ion concentration in the aqueous system as a function of the control difference.

The system according to the invention thus enables fully automatic control, in which an adjustable control difference (deviation of the actual value from the setpoint value of the individual ions) can cause adjustable readjustment of dosing quantities of dosing pumps.

Further objectives, features, advantages and possible applications result from the following description of embodiments not limiting the invention on the basis of the figures. In this context, all the features described and/or illustrated constitute the subject-matter of the invention, either individually or in any combination, even irrespective of their summary in the claims or their relation back. The figures show:

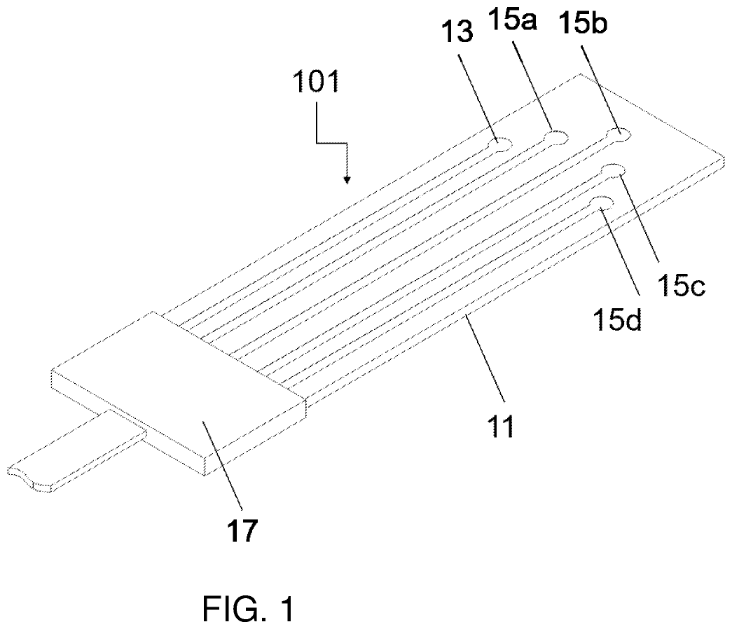

FIG. 1 a schematic drawing of the ion-selective sensor unit 101 according to one embodiment of the invention,

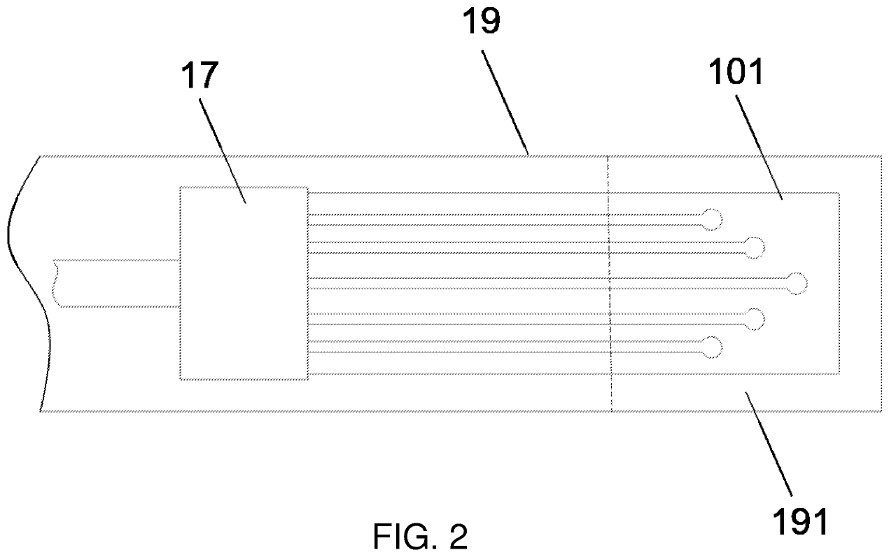

FIG. 2 a schematic drawing of the multi-ion sensor 1 according to an embodiment of the invention in an opened state,

FIG. 3 a schematic drawing of the multi-ion sensor 1 according to one embodiment of the invention in the closed state, and

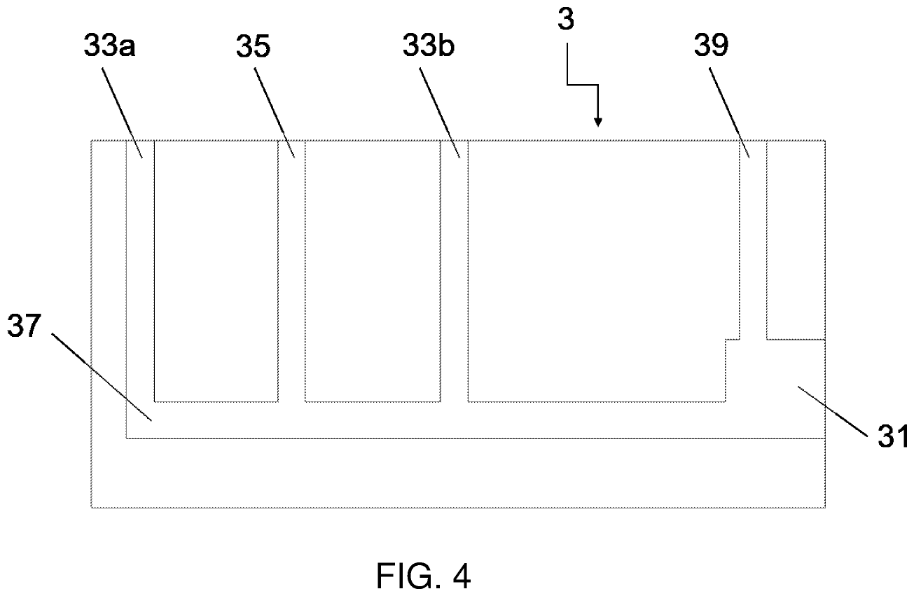

FIG. 4 a schematic sectional view of the flow measuring cell 3 according to an embodiment of the invention.

FIG. 1 schematically shows the ion-selective sensor unit 101 in a preferred embodiment of the invention. The electrodes are mechanically printed on the substrate 11, in this case a flexible printed circuit board, for example as graphite tracks. In the present embodiment, four of the printed electrodes are designed as working electrodes 15a, 15b, 15c, 15d, whereby the majority of the conductive tracks are covered with a lacquer for protection and essentially the end shown round in the figure forms the measuring point. Depending on the ions to be measured, the measuring points are formed accordingly with an ion-sensitive material.

The fifth electrode in this illustration represents the real reference electrode 13, which also initially consists of a printed conductive path (e.g. graphite) with regard to its lead. However, the actual, miniaturised electrode is applied or contacted at the round measuring point, whereby the material consists particularly preferably of polymer-coated fibres to which Ag/AgCl (silver/silver chloride) has been added.

The arrangement or sequence of the reference electrode 13 and the working electrodes 15a, 15b, 15c, 15d in FIG. 1 is merely exemplary and does not define the invention in any specific way.

The ion-selective sensor unit 101 is contacted at the end of the substrate 11 opposite the measuring points by a connection device 17. In a preferred embodiment, the printed conductive tracks have a contact surface at the end with which they can be inserted into the connection device 17. The connection device 17 also has a data cable with a corresponding plug for connection to a computer unit.

FIG. 2 shows how the ion-selective sensor unit 101 with attached connection device 17 is accommodated in the sensor housing 19, whereby the sensor housing 19 is shown open here. The sensor housing 19 may be of one-piece construction, but in a preferred embodiment, which is also shown here, the sensor housing 19 is of two-piece construction, which makes assembly somewhat easier.

The supporting part 191 of the sensor housing 19 is shown with a dashed line, on which the front part of the ion-selective sensor unit 101 rests with its rear side, the measuring points are exposed towards the front. This part of the housing or of the ion-selective sensor unit 101 is washed around or washed over by the liquid to be measured, while the part shown here to the left of the dashed line is firmly and tightly sealed.

FIG. 3 schematically shows the multi-ion sensor 1 of the preferred embodiment of the invention in a closed state. Here the ion-selective sensor unit 101 is shown dashed, which rests on the supporting part 191 of the sensor housing 19. Opposite it, the measuring head 103 is formed by a recessed partial surface 193, so that the measuring gap S can be formed between them. The width B and the height H are shown.

In FIG. 4, the flow measuring cell 3 according to the invention is schematically shown in section according to an embodiment of the invention. In principle, one calibration supply line 33, through which two calibration liquids can be introduced, is sufficient.

However, in practice it has been found to be advantageous to provide a separate calibration supply lines 33a, 33b for each of the two calibration liquids. In the present embodiment, the sample supply line 35 is located between the two calibration supply lines 33a, 33b, but this does not necessarily have to be the case. Depending on the requirements, a further sample supply line 35 could also be provided. The supply lines 33a, 33b, 35 are brought together in the collecting line 37 and led to the receiving opening 31.

This receiving opening 31 is designed to positively receive the measuring head 103 of the multi-ion sensor 1 so that no liquid escapes from the flow measuring cell 3 at this point. The measuring gap S, together with the rear wall of the receiving opening 31, forms a flow measuring space in which a laminar flow can be formed. This is supported by the fact that the collecting line 37 leads into the receiving opening 31 at the bottom, while the drain line 39 is arranged above it.

The present invention is for measuring ions in aqueous systems, in particular, but not limited thereto, for monitoring ion concentrations in aquariums, in this case especially seawater aquariums.

The system for measuring ions in aqueous systems and for adjusting the ion contents in the aqueous systems comprises the flow measuring cell 3 according to the invention with a multi-ion sensor 1 according to the invention installed therein. A pump unit is connected on the input side to the aqueous system for taking the sample, here specifically for example to an aquarium. Dosing pumps are used which can deliver small volumes very precisely.

The hoses from the aquarium to the measuring system in the measuring cell usually contain several millilitres of sample, i.e. in this case aquarium water. Before a measurement, fresh sample water should be added to obtain a current value. In order not to waste the water present in the hoses by discharging it as waste water, it is provided that first a predetermined amount of sample water is pumped through the hoses, whereby the water previously standing in the hoses is returned to the aquarium. Subsequently, the flow of the sample water can be directed into the measuring system, specifically into the flow measuring cell 3, via a change-over valve.

Since each ion-selective electrode drifts over time, calibration is performed before each measurement to ensure reliable measurement. For this purpose, a first calibration liquid is passed through the flow measuring cell 3, the concentration of which is below the expected concentration of the sample water to be measured. Next, the sample water is then passed through the flow measuring cell 3 and the corresponding measured values are recorded as a measurement bundle. Finally, a second calibration liquid is passed through the flow measuring cell 3, the concentration of which is above the expected and to be measured concentration of the sample water.

The liquid volumes of the two calibration liquids and the measured sample water are discarded and added to the waste water. After the measurement is completed, the flow is stopped without the need to empty the flow measuring cell 3 and without the multi-ion sensor 1 falling dry. Herein lies another great advantage of the present invention.

### REFERENCE SIGNS

1 Multi-ion sensor
11 substrate
101 ion-selective sensor unit
103 measuring head
13 reference electrode
15 working electrodes
17 connection device
19 sensor housing
191 supporting part
193 recessed partial surface
3 flow measuring cell
31 receiving opening
33 calibration supply line
35 sample supply line
37 collector line
39 drain line
B width
H height
S measuring gap

1. A multi-ion sensor (1) for measuring ions in aqueous systems comprising
a flexible or semi-flexible substrate (11),
a reference electrode (13) applied to the substrate (11),
at least three working electrodes (15a, 15b, 15c) applied to the substrate (11),
wherein the substrate (11), the reference electrode (13) and the working electrodes (15a, 15b, 15c) form an ion-selective sensor unit (101),
a connection device (17) for the ion-selective sensor unit (101) and
a sensor housing (19) which accommodates an assembly of ion-selective sensor unit (101) and connection device (17),
wherein the reference electrode (13) has dimensions of max. 4 mm×4 mm×4 mm.

2. The multi-ion sensor (1) according to claim 1, wherein the material of the reference electrode (13) is selected from polymer-coated fibres and Ag/AgCl.

3. The multi-ion sensor (1) according to claim 1, wherein the sensor housing (19) accommodates the assembly of ion-selective sensor unit (101) and connection device (17) such that the rear side of the substrate (11) rests on a supporting part (191) of the sensor housing (19), and wherein the sensor housing (19) encloses the assembly of ion-selective sensor unit (101) and connection device (17) to such an extent that the ends of the reference electrode (13) and the working electrodes (15a, 15b, 15c) are exposed with respect to the outside and thus form the measuring head (103) of the multi-ion sensor (1).

4. The multi-ion sensor (1) according to claim 3, wherein the sensor housing (19) has a recessed partial surface (193) which corresponds geometrically with the supporting part (191) with an ion-selective sensor unit (101) resting thereon, so that a measuring gap (S) is formed between the ion-selective sensor unit (101) on the supporting part (191) and the recessed partial surface (193).

5. The multi-ion sensor (1) according to claim 4, wherein the height (H) of the measuring gap (S) and the width (B) of the measuring gap (S) are in a geometric ratio H:B of 1:1 to 4:1.

6. A flow measuring cell (3) for measuring ions in aqueous systems, comprising
a receiving opening (31) for mounting a multi-ion sensor (1) according to of claim 1,
wherein the measuring head (103) of the multi-ion sensor (1) can be mounted in a form-fitting manner in the receiving opening (31) and together with the rear wall of the receiving opening (31) can form a flow-through measuring chamber,
at least one calibration supply line (33),
at least one sample supply line (35),
a manifold (37) receiving the at least one calibration supply line (33) and the at least one sample supply line (35) and extending towards the receiving opening (31)
a drain line (39) which connects upstream of the receiving opening (31).

7. The flow measuring cell (3) according to claim 6, wherein the diameters of the calibration supply line (33) and/or the sample supply line (35) and/or the collecting line (37) are 2 mm to 4 mm.

8. The flow-through measuring cell (3) according to claim 6, wherein the cumulative volume of the calibration supply line (33), the sample supply line (35), the collecting line (37) and the flow-through measuring chamber is 2 ml to 5 ml.

9. A method for measuring ions in aqueous systems comprising the steps of
a) providing a flow-through measuring cell (3) according to claim 6 with an assembled multi-ion sensor (1) according to claim 1,
b) introducing a first aqueous calibration solution into the flow measurement cell (3) with flushing of the ion-selective sensor unit (101) of the multi-ion sensor (1), thereby performing a first calibration and recording a first reference measurement value,
c) introducing an aqueous sample into the flow measuring cell (3) with flushing of the ion-selective sensor unit (101) of the multi-ion sensor (1), thereby recording a measurement value bundle,
d) introducing a second aqueous calibration solution into the flow measuring cell (3) with flushing of the ion-selective sensor unit (101) of the multi-ion sensor (1), thereby carrying out a second calibration and recording a second reference measured value,
wherein steps a), b) c) and d) are carried out quasi-continuously without draining the flow measuring cell (3),
e) calculating the measurement values from first and second reference measurement values and measurement value bundles,
f) processing the measurement values in a computing unit.

10. The method according to claim 9, wherein the volume of the first aqueous calibration solution, the aqueous sample and the second aqueous calibration solution required for the measurement is 3 ml to 10 ml.

11. The method according to claim 9, wherein before step b) the volume of liquid present in the feed lines to the flow-through measuring cell (3) is completely replaced by fresh volume of liquid of the aqueous system, the replaced volume of liquid present being returned to the aqueous system.

12. A system for measuring ions in aqueous systems and for adjusting the ion contents in the aqueous systems, comprising
a flow-through measuring cell (3) according to claim 6,
a multi-ion sensor (1) mounted in the flow measuring cell (3) according to claim 1,
a pump unit which is fluid-dynamically connected on the input side to the aqueous system and to storage containers for the first and second aqueous calibration solution for taking the sample and which is fluid-dynamically connected on the output side to the at least one calibration supply line (33) and to the at least one sample supply line (35),
a control unit which is electronically connected on the input side to the multi-ion sensor (1),
the control unit having evaluation electronics for processing the first and second reference measured values and measured value bundles.

13. The system according to claim 12, wherein the control unit is designed to regulate the ion concentration in the aqueous system as a function of the control difference.
